(19) United States
(12) Patent Application Publication
(10) Pub. No.: US 2026/0127737 A1
(43) Pub. Date: May 7, 2026

(54) PERIPHERAL SCREW LENGTH DETERMINATION

(71) Applicant: Depuy Synthes Products, Inc., Raynham, MA (US)

(72) Inventors: Nicolas Demanget, Saint-Egrève (FR); Alexander Samaha, Allston, MA (US); Cory Emil, Milton, MA (US); Cyprien Adnet, Bourg-lès-Valence (FR)

(21) Appl. No.: 18/939,117

(22) Filed: Nov. 6, 2024

(51) Int. Cl. G06T 7/00; A61B 34/10; A61B 34/20

(52) U.S. Cl. CPC G06T 7/0012; A61B 2034/102; A61B 2034/2046

(57) ABSTRACT

Systems, methods, and devices are disclosed comprising a computer-assisted surgical system for an implant that is configured to be affixed by a plurality of peripheral screws to a bone of a patient. The computer-assisted surgical system comprising an instrument and a navigation array attached to the instrument, the instrument for cutting the bone, a tracking system to detect and track elements of the navigation array, a display, and a controller having at least one processor configured to receive an image of the bone for receiving the implant, receive instrument positional data from the tracking system to determine a trajectory of the instrument, generate, on the display, a representation of a distal end of the instrument overlaid over the image, generate, on the display, a representation of a scale along the trajectory of the instrument overlaid over the image, wherein the scale includes graduations related to a length of a peripheral screw along the trajectory, and wherein the scale graduations are independent of the position of the distal end of the instrument, and update at least one of the representations as the instrument moves.

### BACKGROUND

Many orthopedic surgeries involve implant systems. For example, shoulder arthroplasties may require replacing an articulation surface in a joint. In this example, a baseplate (such as a glenoid reconstruction reverse baseplate) is secured to bone by a plurality of peripheral screws. Peripheral screws are important for both strong fixation and anti-rotation of the baseplate.

Peripheral screws come in a number of predetermined lengths. As may be appreciated, a surgeon performing such a procedure may desire to select a trajectory that will maximize fixation and/or a peripheral screw length that is sufficiently long yet avoids compromising the bone into which the peripheral screw will be placed (e.g., to avoid a peripheral screw that is too long). Baseplate depth, inclination, retroversion, and position of openings for receiving the peripheral screws all may be known but given that a drill trajectory occurs in three-dimensions, determination of clinically desirable peripheral screw length (and thus depth into the bone) from two-dimensional images (even where a plurality of views are present) is difficult. Moreover, making an informed decision about peripheral screw length relative to its final seating in the baseplate can be challenging when merely viewing imaging.

Accordingly, there is a need for determining peripheral screw trajectory (e.g., from an opening in the baseplate for receiving the peripheral screw to a distal end of the bore) and/or desirable peripheral screw length. It is also desirable to provide a graphical user interface that renders visualization of screw length more intuitive.

### SUMMARY

Systems, methods, and devices are disclosed comprising computer-assisted surgical systems for an implant that is configured to be affixed by a plurality of peripheral screws to a bone of a patient. The computer-assisted surgical systems may comprise an instrument and a navigation array attached to the instrument, the instrument for cutting the bone, a tracking system to detect and track elements of the navigation array, a display, and a controller having at least one processor configured to receive an image of the bone for receiving the implant, receive instrument positional data from the tracking system to determine a trajectory of the instrument, generate, on the display, a representation of a distal end of the instrument overlaid over the image (e.g., the image may be a 2D slice of a 3D volume), generate, on the display, a representation of a scale along the trajectory of the instrument overlaid over the image, wherein the scale includes graduations related to a length of a peripheral screw along the trajectory, and wherein the scale graduations are independent of the position of the distal end of the instrument, and update at least one of the representations as the instrument moves. In some embodiments, the controller is further configured to receive a position of the bone for receiving the implant. For example, a second navigation array may be attached to the patient, and the tracking system used to detect and track elements of the second navigation array to determine the position of the bone.

As will be described, a controller may overlay a representation of a scale and/or the scale and a drill position (e.g., as an overlay image) over an image of the patient to allow a user (e.g., surgeon) to determine a clinically desirable peripheral screw length.

### BRIEF DESCRIPTION OF THE DRAWINGS

FIG. 1A shows a schematic of a glenoid reconstruction reverse baseplate with superimposed theoretical points for centers of rotation for the peripheral screws according to an embodiment of the present application.

FIG. 1B shows a schematic of a section of the glenoid reconstruction baseplate of FIG. 1A where the superimposed theoretical points for centers of rotation are fully seated in the baseplate and have been used to derive a plane relative to a coordinate system of the baseplate.

FIG. 2A shows an exemplary display comprising representations of the baseplate, the derived plane, a scale, and a drill overlaid over a patient image.

FIG. 2B shows another exemplary display comprising representations of the baseplate, the derived plane, a scale, and a drill overlaid over another patient image.

FIG. 2C shows another exemplary display comprising representations of the baseplate, the derived plane, the scale, and the drill overlaid over a patient image, wherein the drill depth has changed as compared to FIG. 2A.

FIG. 2D shows another exemplary display comprising representations of a scale and a drill overlaid over a pair of patient images.

FIG. 3 shows a flowchart of a method of determining the derived plane.

FIG. 4 shows a flowchart of a method of generating the representation of the scale.

FIG. 5A shows another exemplary display comprising representations of the baseplate, a scale, and the drill overlaid over a patient image.

FIG. 5B shows another exemplary display comprising representations of the baseplate, the scale, and the drill overlaid over another patient image.

FIG. 5C shows another exemplary display comprising representations of a baseplate, a scale, and a drill overlaid over another patient image.

FIG. 6 shows a flowchart of a method of generating the representations of the scale and the drill according to another embodiment.

### DETAILED DESCRIPTION

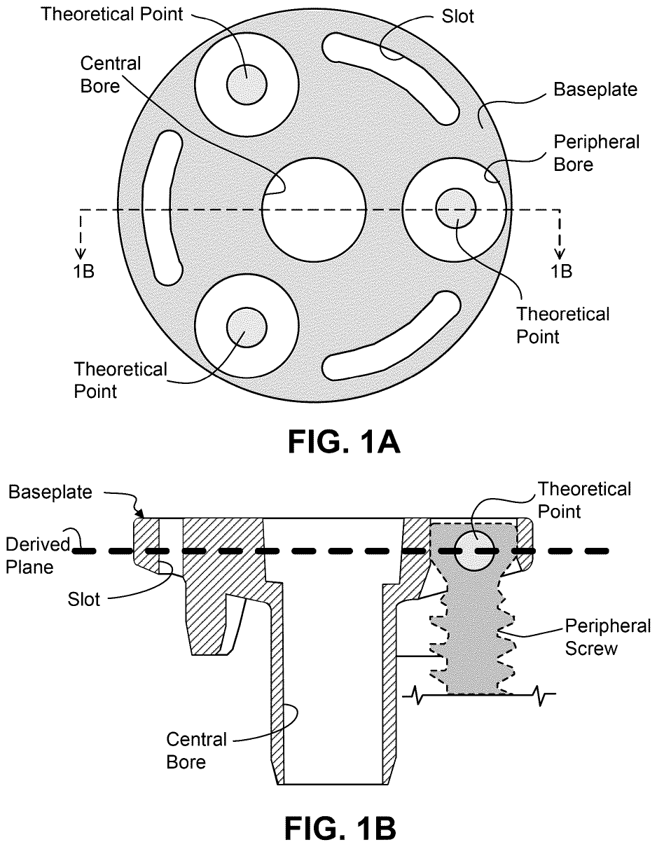

FIG. 1A shows a schematic of a glenoid reconstruction baseplate. Typically, such baseplates comprises a flat surface having a plurality of slots and peripheral bores, the peripheral bores for receiving peripheral screws to affix the baseplate to a bone (not depicted). A central bore is provided and is typically threaded to receive additional components, such as a central fixation element and/or a glenosphere. This description uses, as an example, a particular surgery and implant type, but it will be recognized that the systems and methods described herein are not limited to these applications. For example, principles described herein may be applied more generally to applications that include knee surgery, hip surgery, spine surgery, etc. The controller systems described herein may be utilized in various applications involving robotic, robot-assisted, Augmented Reality (AR), Virtual Reality (VR), Mixed Reality (MR), and non-robotic systems for surgical approaches. A computer-assisted surgical system that may be used in the following embodiments may have a global coordinate system, a patient coordinate system, an instrument coordinate system, and/or an implant coordinate system. As can be appreciated, positions of the instrument or implant can be determined with respect to the patient, or more importantly, to a bone of the patient.

Returning to FIG. 1A, each peripheral bore has a theoretical point for a center of rotation for a peripheral screw. By “theoretical” it is meant that this point relates to the coordinate system of the baseplate and is described by the theoretical points of the center of rotation of the peripheral screws when fully seated in the baseplate (FIG. 1B). The theoretical points may be used to derive a plane relative to a coordinate system of the baseplate (dashed line in FIG. 1B). Accordingly, the derived plane is dependent on the morphology of the baseplate (at least with respect to the theoretical points of the center of rotation of the peripheral screws, which relates to a thickness of the baseplate).

Preferably, the depth of the baseplate is known (e.g., as the depth of the baseplate has an effect on the derived plane). As may be appreciated, a derived plane may be specific to an implant type. In a non-limiting example of a glenoid reconstruction reverse baseplate, for each peripheral hole of a plurality of holes, a point is defined using a position of the center of the screw head when the screw is in place in the baseplate, and these multiple points define the derived plane. In some embodiments, a best fit through theoretical points of a particular implant may be used.

In some embodiments, a central axis of a baseplate (such as a glenoid baseplate) is known. The central axis may be used for calculating a rotational orientation (e.g., clocking) of the baseplate which may be useful when navigating the peripheral screw trajectory. It is understood that a derived plane may be determined without knowing the central axis.

In operation, a bore is placed in the patient's bone and the implant impacted in place. At this point, the implant is retained without the peripheral screws. Then a central screw is inserted through the implant into the scapula and tightened. Then, peripheral screw pilot holes will need to be drilled and peripheral screws inserted.

A series of images are obtained (e.g., retrieved). The image data preferably comprises pre-operative images, that is, images acquired before the surgery (e.g., before the implant is positioned). In a preferred embodiment, the image data used in navigation is based on pre-operative CT scans. Stated differently, the techniques described herein do not rely on intra-operative fluoroscopy. The image data may comprise computed tomography (CT), magnetic resonance imaging (MRI), or other three-dimensional (3D) images. The image data may comprise unprocessed data or may be processed or segmented to show boundaries between different tissues (bone, nerve, muscle, etc.). The image data may comprise two-dimensional (2D) images, such as X-rays, or 2D images merged to afford a 3D image (e.g., fluoroscopy), or video images (e.g., from a TELIGEN™ camera, endoscopes, microscopes, etc.). The image data may be provided to the controller in a predetermined format, such as Digital Imaging and Communications in Medicine (DICOM) format.

Preferably, the image data comprises 2D slices of images of the patient's bone, such as a stack of 2D slices using DICOM image format (CT Scan). The image data may be referred to herein as a 2D slice of a 3D volume.

A controller receives the image data. The controller typically comprises a power supply, AC/DC converters, motion controllers, fuses, real-time control system interface circuits, and other components typically included in computer-assisted surgical systems. The controller is configured to render displays which comprised image data with overlaid representations.

In some embodiments, the derived plane serves as the beginning point for a representation of a scale overlaid on a 2D slice of a 3D volume. The representation of the scale and the overlay are generated by the controller and displayed on a display. The scale may comprise graduations. The graduations may be indicative of units of distance, such as millimeters, or indicative of sizes of commercially available peripheral screws. As will be explained, the scale is not simply a legend corresponding to the image but is associated with a live, navigated drill trajectory. As the trajectory of the instrument (e.g., a drill) changes, the trajectory of the scale moves with it. For example, as the instrument pivots, the scale moves with the instrument. In some embodiments, the scale is displayed as floating, for example, the controller is further configured to display the scale as free to move in space until a determination is made by the controller that the drill (e.g., drill bit) is in position, and then the controller is further configured to display the initial graduation of the scale as locked in position (e.g., at the derived plane) while allowing the representation of the scale to pivot.

A point at which a distal margin of bone overlaid on the image intersects with the scale is important for providing the user (e.g., a surgeon) with the information needed to select a screw length. A user can then determine an appropriately sized peripheral screw, for example, by inspecting the scale and determining an appropriate length (for example, an appropriate length is determined where the scale graduation linked to the drill axis meets the cortical bone posteriorly). As noted above, the scale may be indicative of a predetermined length and/or a size of available peripheral screws.

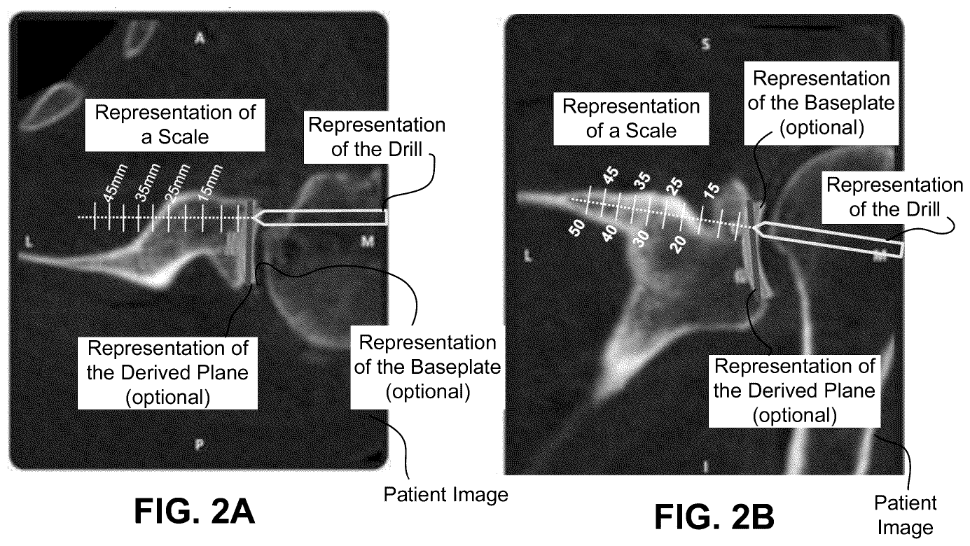

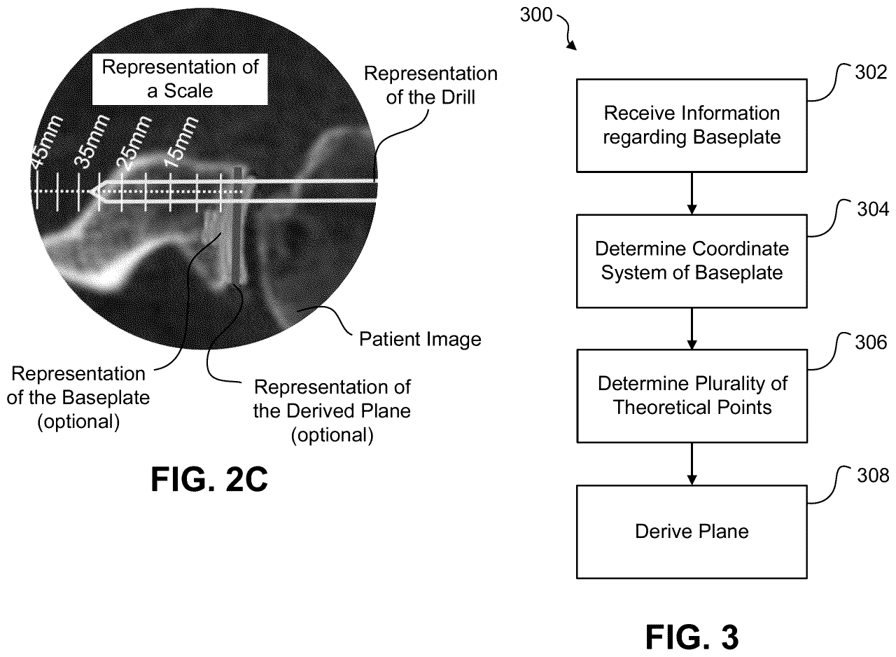

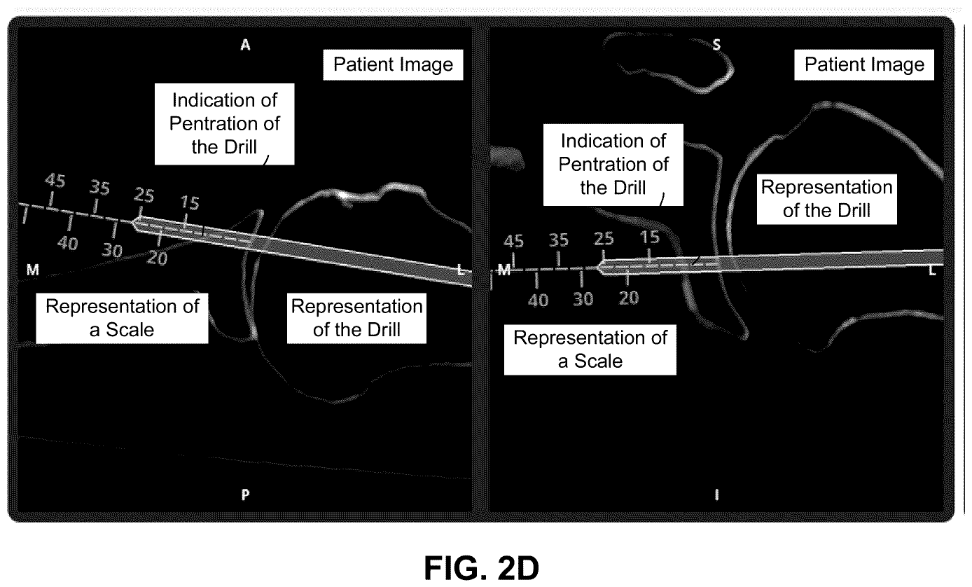

FIG. 2A shows an exemplary display generated by the controller comprising representations of a scale and an instrument (e.g., a drill tip) overlaid over a patient image. As can be appreciated, the representations are elements of a graphical user interface (GUI) generated by the controller to be displayed (e.g., overlaid) superimposed on an image. Preferably, the image is a 2D slice of a 3D volume using DICOM image format. Stated differently, the representations are GUI elements that are not present in the image. By way of non-limiting example, the representation of the drill may be a rendering based on representative computer aided design (CAD) data. The controller is configured to update the representation of the drill axis (and therefore the scale) as a position of the drill changes. The position of the drill may be detected by a navigation system that is part of, or in communication with, the controller.

For ease of explanation, FIG. 2A is shown as an exemplary display generated by the controller further comprising representations of the baseplate and the derived plane. Both are optional for display, and in practice, representations of the baseplate and/or the derived plane may not be generated by the controller. The derived plane may serve as the locked/anchored starting point for the scale. It may be appreciated that some representations relate to tangible objects not visible in the image (e.g., the drill and the (optionally displayed) baseplate) and some of the representations are not tangible, but are visually perceptible to a user viewing the display (e.g., the scale and the (optionally displayed) derived plane).

The representation of the scale is displayed (e.g., always displayed) as aligned with the trajectory of the drill. In some embodiments, the derived plane serves as the beginning point for the representation of the scale. In some embodiments, the scale is displayed as free to move in space (floating) until a determination is made by the controller that the drill (e.g., drill bit) is in position, and then the controller locks (e.g., anchors) the representation of the scale as beginning at the base plate but free to pivot about the datum (as depicted in FIG. 2B).

FIG. 2B is similar to FIG. 2A but with the representations overlaid over a different slice (e.g., a different 2D slice of a 3D volume) in response to a detected change of orientation of the drill (as will be explained). FIG. 2B shows the controller's response to a change in drill trajectory (such as by pivoting about the datum during navigation (e.g., before entering the bone (FIG. 2C)). In this embodiment, the graduations of the scale represent predetermined peripheral screw sizes.

To determine the pose or trajectory of the drill, the position and orientation of the drill is determined and/or updated. To determine positional information of the drill relative to the patient, surgical navigation or tracking may be employed. Examples of tracking systems include optical tracking systems with reflective markers, radio frequency (RF) tracking systems, electromagnetic interference (EMI) tracking systems, visual systems including for example chest trackers, Aruco markers, machine vision using shape recognition, etc.

For example, optical navigation or tracking systems may utilize stereoscopic sensors to detect infra-red (IR) light reflected or emitted from one or more optical markers affixed to surgical instruments and/or portions of a patient's anatomy. A navigation array or tracker having a unique constellation or geometric arrangement of reflective elements may be coupled to a surgical instrument and, once detected by stereoscopic sensors, the relative arrangement of the elements in the sensors'field of view, in combination with the known geometric arrangement of the elements, may allow the system to determine a three-dimensional position and orientation of the tracker and, as a result, the instrument and/or anatomy to which the tracker is coupled.

Accordingly, a tracker may be mounted on the drill (e.g., integrally or removably). The tracker may be an optical tracker comprising reflective markers that may be detected by a navigation system. The tracker may comprise light emitting diodes (LEDs) as markers. The tracker may comprise RF trackers as markers. The tracker may comprise electromagnetic or EMI trackers as markers. The markers of the tracker may have a specific fixed geometric relationship such that they define a constellation, thereby indicating the pose of the drill.

Optionally, a second tracker having a fixed geometric relationship may be coupled to a portion of patient anatomy, a surgical surface, or other immobile component. The second tracker may employ the same types of markers as the drill tracker. The second tracker may employ different types of markers as the drill tracker. The second tracker may represent a global coordinate system, for example, with reference to the patient. In some embodiments, the drill tracker is active (e.g., moving and detected at regular intervals) and the patient tracker is passive (e.g., almost immobile, given the context that bones are never completely immobile).

The navigation system and/or the controller may utilize the known fixed geometric relationship between the trackers to determine a precise three-dimensional position and orientation of the drill (e.g., and therefore the trajectory). While the pose of the drill is important for determining an axis (e.g., in order to display the representation of the scale), in some embodiments, it is also useful for determining progress of the drill bit (e.g., as a method of determining bit depth).

FIG. 2C shows another exemplary display comprising representations of the baseplate, the derived plane, the scale, and the drill overlaid over a patient image, wherein the drill depth has changed as compared to FIG. 2A. The scale graduations are independent of the position of the drill tip. Stated differently, the beginning of the scale is dependent on derived plane (which may or may not be displayed as a representation). As long as the drill tip is moved along the scale keeping the same trajectory, the same image is used. If the trajectory of the drill is changed, the controller is configured to change the generated display so that the representations are overlaid over a different slice (e.g., a different 2D slice of the 3D volume). As the drill advances, the representations support selection of an appropriate screw length by the user comparing the drill position to the scale graduations.

FIG. 2D shows another exemplary display comprising representations comprising representations of a scale and a drill overlaid over a pair of patient images. The controller may be further configured to display an indication of penetration of the drill bit into the bone, for example, by depicting the representation of the drill overlapping along the axis of the representation of the scale. The controller may be further configured to display an indication of penetration of the drill bit into the bone, for example, by depicting the representation of the drill overlapping along the axis of the representation of the scale and by adding a color change to the overlapped portion to aid a user in determining how far the drill bit has penetrated the bone.

FIG. 3 shows a flowchart of a method 300 of determining the derived plane (which may or may not be displayed as a representation by the controller, but is useful for determining the scale start position or beginning of the graduations of the scale). At step 302, the controller is configured to receive information about the implant, that is, the baseplate of the implant. Examples of such information may be type, thickness, peripheral bore spacing, size, number of holes, etc.

At step 304, the controller is optionally configured to determine a coordinate system of the baseplate. At step 306, the controller is configured to determine theoretical points of the center of rotation of the peripheral screws when the peripheral screws are fully seated in the baseplate (based on the information). At step 308, the controller is configured to determine a derived plane of the baseplate based on the theoretical points. The derived plane is useful, for example, the controller may be configured to generate the representation of the scale using the derived plane as the beginning of the graduations of the scale. The graduations may represent units of distance and/or the graduations may represent predetermined peripheral screw sizes. The controller may be configured to provide a user interface to toggle between the graduation types.

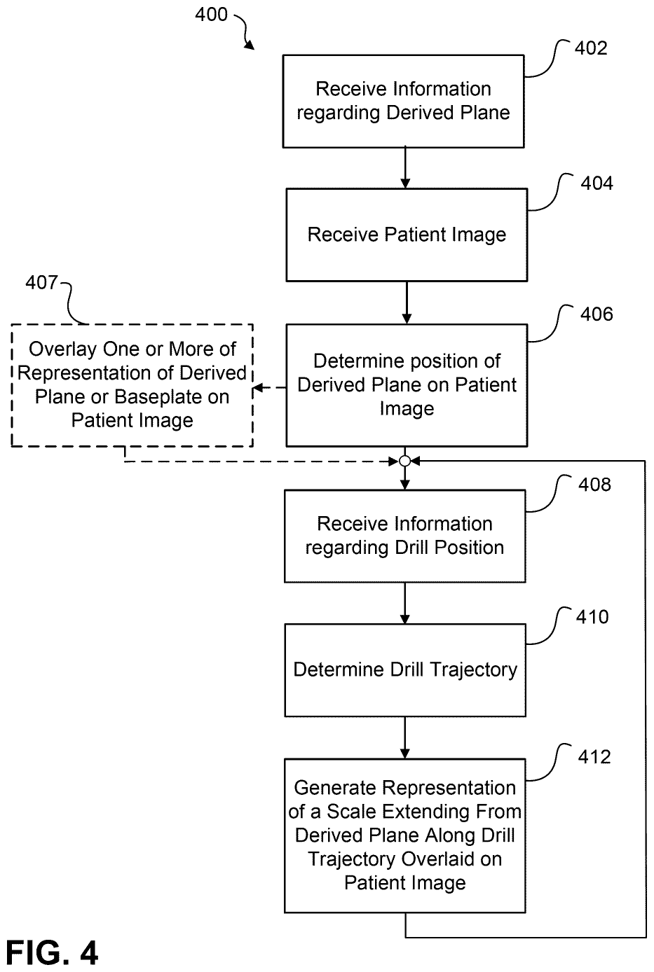

FIG. 4 shows a flowchart of a method 400 of generating the representation of the scale. At step 402, the controller receives information regarding the derived plane. At step 404, the controller receives a patient image, for example, a 2D slice of a 3D volume. At step 406, the controller determines a position where the derived plane would fall on the patient image, e.g., to determine a position for a beginning point for a representation of a scale.

Optionally, at step 407, the controller may be configured to overlay a representation of the derived plane on the patient image. It is understood that step 407 may be omitted. Not depicted, but also optionally, the controller may be configured to overlay a representation of the baseplate on the patient image. Alternatively, the controller may be configured to overlay a representation of the derived plane and a representation of the baseplate on the patient image.

At step 408, the controller receives information (e.g., such as tracking information) regarding the drill position. At step 410, using the drill position, the controller determines a drill trajectory. At step 412, the controller generates a representation of a scale extending from the derived plane along the drill trajectory and overlays the representation on the patient image. The drill position may be changed by the user, and the controller may perform steps 408-412 again. As long as the drill tip is moved along the scale keeping the same trajectory, the same image is used. If the trajectory of the drill is changed, such as by pivoting, the representations of the drill and the scale are overlaid over a different slice (e.g., a different 2D slice of a 3D volume).

In another embodiment, the controller may be further configured to suggest a desirable screw length. The controller may be configured to consider one or more of drill position, baseplate position, or work (e.g., time, current, or torque) of drilling to determine a best fit screw length.

For example, the controller may be configured to monitor work required to drill the bone. The controller may be configured to monitor a torque required to drill the bone. The controller may be further configured to associate a determined torque with a type of bone, for example, to determine when the drill tip is drilling in cortical bone or cancellous bone. The controller may be configured to determine a distance from the base plate (or the derived plane) to the cortical bone. The controller may be configured to monitor a current associated with one or more motors of the drill. The controller may be further configured to associate a determined current with a type of bone, for example, to determine when the drill tip is drilling in cortical bone or cancellous bone. The controller may be configured to determine a distance from the base plate (or the derived plane) to the cortical bone.

In some embodiments, the controller may be configured to allow adjustment to the position of the derived plane, for example, based on a determined position of the cortical bone (for example, based on work required to drill the bone).

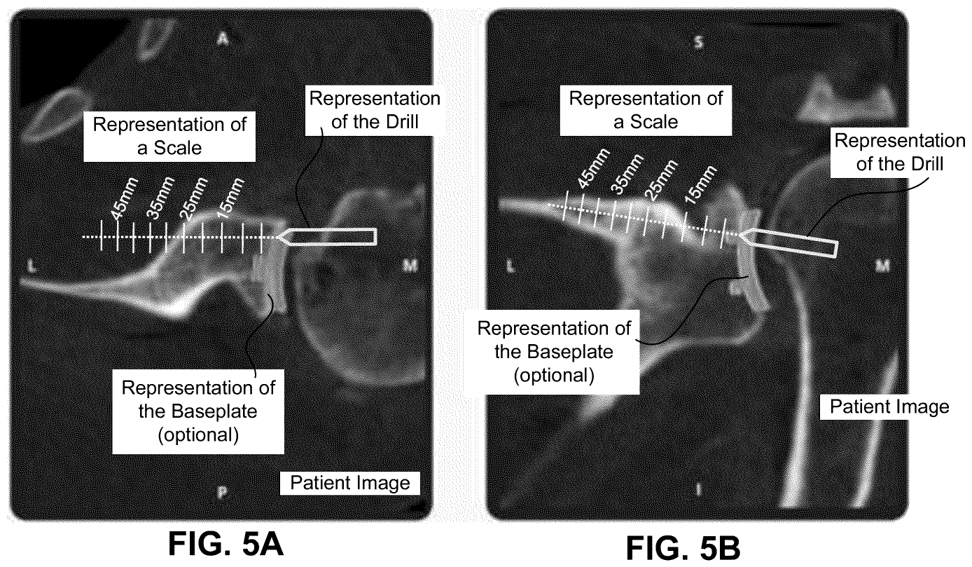

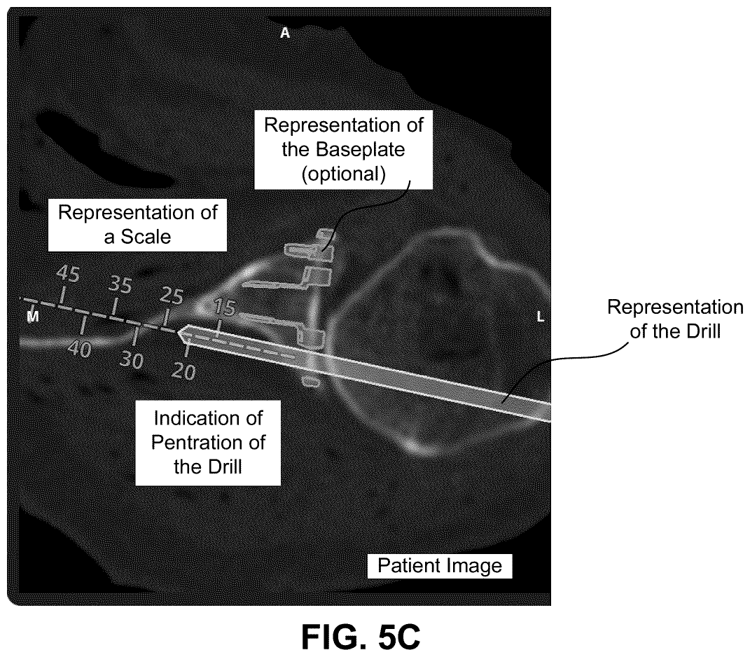

FIG. 5A shows another exemplary display comprising representations of the baseplate (optional), a scale, and the drill overlaid over a patient image. This embodiment is similar to FIG. 2A, but no representation of the derived plane is displayed by the controller. As with previous embodiments, the scale and drill have the same trajectory, but the scale graduations are independent of the position of the drill tip (e.g., the graduation beginning is still at the intersection of the drill trajectory and the derived plane (not depicted). The graduations may represent units of distance (as depicted) and/or the graduations may represent predetermined peripheral screw sizes. The controller may be configured to provide a user interface to toggle between the graduation types.

FIG. 5B shows another exemplary display illustrating the controller's response to a change in drill trajectory (such as by pivoting about the datum during navigation (e.g., before entering the bone). For example, the representations of the baseplate (optional), the scale, and the drill are overlaid over another patient image, for example, over a different slice (e.g., a different 2D slice of a 3D volume).

FIG. 5C shows another exemplary display comprising representations of the baseplate (optional), a scale, and the drill overlaid over a patient image. As with previous embodiments, the scale graduations are independent of the position of the drill tip. The graduations may represent units of distance and/or the graduations may represent predetermined peripheral screw sizes (as depicted). The controller may be configured to provide a user interface to toggle between the graduation types. As the drill bit enters the bone, the representation of the drill tip is moved along the scale. If the drill keeps the same trajectory, the same image is used. If the trajectory of the drill is changed, the representations are overlaid over a different slice (e.g., a different 2D slice of a 3D volume).

As the drill advances, the representations support selection of an appropriate screw length by the user comparing the drill position to the scale graduations, for example, the user (e.g., surgeon) checks visually where the drill bit trajectory intersects the posterior cortical bone and reads the associated graduation. In another embodiment, the controller may be configured to suggest an appropriate screw length. The controller may suggest the appropriate screw length further considering one or more of drill position, baseplate position, or work (e.g., time, current, or torque) of drilling to determine a best fit screw length.

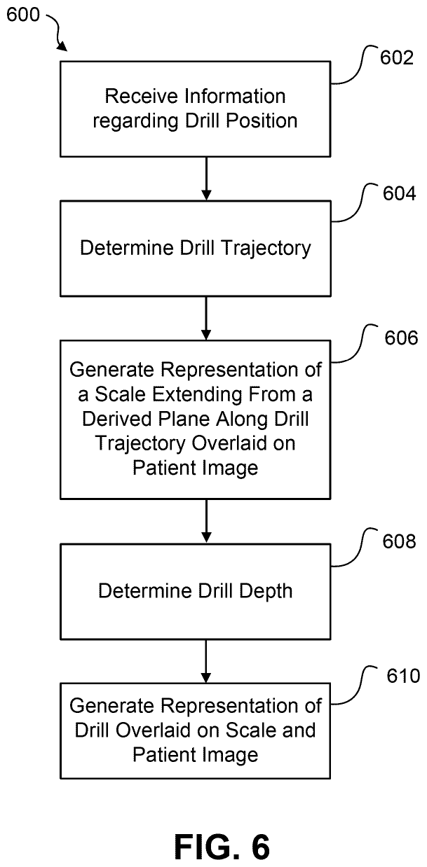

FIG. 6 shows a flowchart of a method 600 of generating the representations of the scale and the drill. Exemplary displays may include those depicted in FIGS. 2C, 2D, and 5C.

The controller may have already received a patient image, for example, a 2D slice of a 3D volume. At step 602, the controller receives information (e.g., such as tracking information) regarding the drill position. At step 604, using the drill position, the controller determines a drill trajectory.

At step 606, the controller generates a representation of a scale extending from the derived plane (discussed above) along the drill trajectory and overlays the representation on the patient image. At step 608, the drill depth is determined. At step 610, a representation of the drill is overlaid on the scale and patient image. As long as the drill tip is moved along the scale keeping the same trajectory, the same image is used. If the trajectory of the drill is changed, the representations are overlaid over a different slice (e.g., a different 2D slice of the 3D volume).

In a first example, a computer-assisted surgical system for an implant is provided that is configured to be affixed by a plurality of peripheral screws to a bone of a patient. The computer-assisted surgical system comprising an instrument and a navigation array attached to the instrument, the instrument for cutting the bone, a tracking system to detect and track elements of the navigation array, a display, and a controller having at least one processor configured to receive an image of the bone for receiving the implant, receive instrument positional data from the tracking system to determine a trajectory of the instrument, generate, on the display, a representation of a distal end of the instrument overlaid over the image, generate, on the display, a representation of a scale along the trajectory of the instrument overlaid over the image, wherein the scale includes graduations related to a length of a peripheral screw along the trajectory, and wherein the scale graduations are independent of the position of the distal end of the instrument, and update at least one of the representations as the instrument moves.

In some embodiments, the image is a 2D slice of a 3D volume.

In some embodiments, the controller is further configured to receive a position of the bone for receiving the implant. For example, a second navigation array may be attached to the patient, and the tracking system used to detect and track elements of the second navigation array to determine the position of the bone.

In some embodiments, the controller is further configured to receive information about the implant. The controller may be further configured to determine theoretical points of the center of rotation of peripheral screws when the peripheral screws are fully seated in the implant. The controller may be further configured to determine a derived plane of the implant based on the theoretical points.

In some embodiments, the controller is further configured to generate the representation of the scale using the derived plane as the initial graduation of the scale. For example, graduations of the scale may represent units of distance. For example, graduations of the scale may represent predetermined peripheral screw sizes.

In some embodiments, the controller is further configured to generate the representations such that if the distal end of the instrument is moved along the scale, the same image is used, but if the trajectory of the instrument is changed, the representations are overlaid over a different 2D slice of the 3D volume.

In some embodiments, the controller is further configured to generate, on the display, a representation of the implant overlaid over the image.

In some embodiments, the controller is further configured to suggest a peripheral screw length. The controller may suggest the appropriate screw length further considering one or more of drill position, baseplate position, or work (e.g., time, current, or torque) of drilling to determine a best fit screw length.

In some embodiments, the controller is further configured to determine at least one of a torque for the instrument or a time of operation for the instrument, and to use the determined torque or the determined time to determine a best fit peripheral screw length. For example, the controller may be further configured to determine a torque of the instrument and associate the determined torque with a type of bone. For example, the controller may be further configured to determine a current of the instrument and associate the determined torque with a type of bone. In either case, the controller then may determine a position of the associated bone type and use the position of the associated bone type as the initial graduation of the scale.

In some embodiments, if the position and orientation of the implant is known, the controller is further configured to use this information to determine a derived plane of the implant and to use the derived plane as the initial graduation of the scale.

In some embodiments, the controller is further configured to suggest an appropriate peripheral screw based on one or more of a bone type, instrument position, or instrument work (e.g., torque or current).

In some embodiments, the controller is further configured to display the scale as floating, for example, the controller is further configured to display the scale as free to move in space until a determination is made by the controller that the drill bit is in position, and then the controller is further configured to display the initial graduation of the scale as locked in position while allowing the representation of the scale to pivot.

1. A computer-assisted surgical system for an implant that is configured to be affixed by a plurality of peripheral screws to a bone of a patient, comprising:
an instrument and a navigation array attached to the instrument, the instrument for cutting the bone;
a tracking system to detect and track elements of the navigation array;
a display; and
a controller having at least one processor configured to:
receive an image of the bone for receiving the implant;
receive instrument positional data from the tracking system to determine a trajectory of the instrument;
generate, on the display, a representation of a distal end of the instrument overlaid over the image;
generate, on the display, a representation of a scale having a same trajectory as the trajectory of the instrument, at least a portion of the representation of the scale extending past the representation of the distal end of the instrument, the representation of the scale being overlaid over the image, wherein the scale includes graduations related to a length of a peripheral screw along the scale trajectory, and wherein an initial graduation of the scale is independent of the position of the distal end of the instrument; and
update at least one of the representations as the instrument moves.

2. The system of claim 1, wherein the image is a 2D slice of a 3D volume.

3. The system of claim 1, wherein the controller is further configured to receive a position of the bone for receiving the implant.

4. The system of claim 1, wherein the controller is further configured to receive information about the implant.

5. The system of claim 4, wherein the controller is further configured to determine theoretical points of the center of rotation of peripheral screws when the peripheral screws are fully seated in the implant.

6. The system of claim 5, wherein the controller is further configured to determine a derived plane of the implant based on the theoretical points.

7. The system of claim 6, wherein the controller is further configured to generate the representation of the scale using the derived plane as the initial graduation of the scale.

8. The system of claim 7, wherein graduations of the scale represent units of distance.

9. The system of claim 7, wherein graduations of the scale represent predetermined peripheral screw sizes.

10. The system of claim 2, wherein the controller is further configured to generate the representations such that if the distal end of the instrument is moved along the scale, the same image is used, but if the trajectory of the instrument is changed, the representations are overlaid over a different 2D slice of the 3D volume.

11. The system of claim 1, wherein the controller is further configured to generate, on the display, a representation of the implant overlaid over the image.

12. The system of claim 1, wherein the controller is further configured to suggest a peripheral screw length.

13. The system of claim 12, wherein the controller is further configured to determine at least one of a torque for the instrument or a time of operation for the instrument, and to use the determined torque or the determined time to determine a best fit peripheral screw length.

14. The system of claim 1, wherein if the position and orientation of the implant is known, the controller is further configured to use this information to determine a derived plane of the implant and to use the derived plane as the initial graduation of the scale.

15. The system of claim 1, wherein the controller is further configured to determine a torque of the instrument and associate the determined torque with a type of bone.

16. The system of claim 15, wherein the controller is further configured to determine a position of the associated bone type and use the position of the associated bone type as the initial graduation of the scale.

17. The system of claim 1, wherein the controller is further configured to determine a current of the instrument and associate the determined torque with a type of bone.

18. The system of claim 17, wherein the controller is further configured to determine a position of the associated bone type and use the position of the associated bone type as the initial graduation of the scale.

19. The system of claim 1, wherein the controller is further configured to suggest an appropriate peripheral screw based on one or more of a bone type, instrument position, or instrument work.

20. The system of claim 1, wherein the controller is further configured to display the scale as free to move in space until a determination is made by the controller that the drill bit is in position, and then the controller is further configured to display the initial graduation of the scale as locked in position while allowing the representation of the scale to pivot.
